(19) United States
(12) Patent Application Publication
(10) Pub. No.: US 2007/0001905 A1
(43) Pub. Date: Jan. 4, 2007

(54) Detecting the position of X-ray detector

(76) Inventor: Esa Eronen, Littoinen (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/172,606

(22) Filed: Jun. 30, 2005

(51) Int. Cl. G01S 3/02; A61B 5/05

(52) U.S. Cl. 342/463; 600/424

(57) ABSTRACT

A method for determining the position of X-ray detector where the detector is equipped with one or more radio or magnetic means, and one or more radio or magnetic means are in a distance from the detector, and said radio means are used to determine at least either position or angle of the detector in respect to the X-ray source.

The invention relates to measuring or detecting the position of an X-ray detector in an X-ray imaging system.

In medical X-ray projection imaging system it is required that the X-ray beam is accurately focused with the X-ray image detector, film or electronic, to avoid unnecessary patient dose. It is also necessary to control projection angle accurately to achieve maximum clinical information.

Common method to indicate X-ray beam direction and coverage is to project light grid indicating the X-ray beam over the detector plane. Another method is to use mechanical means to position the detector in correct position.

The problem to be solved is how to position the sensor or how to direct the X-radiation, when there is no possibility to use the light grid or mechanically control the imaging geometry. Especially difficult is the situation, when the sensor is wireless and it should be placed inside a non-transparent object cavity without possibility to use any mechanical means to ensure the position. In that case the detector positioning may fail, or a large area is radiated to ensure the right exposure to the detector. Both cases the patient is radiated more than necessary.

The solution is to measure the position and angle of the X-ray detector with radio positioning or with magnetic fields. The principally alike radio technology is already used in navigation or positioning systems, like Decca, GPS, Loran-C, VOR, or VORTAC. Additionally the angle of X-ray detector can be detected by antenna technology, detecting the direction of the electromagnetic field, or by measuring the phase difference of received radio waves with two or more antennas. Compared to the “large scale” positioning systems, the radio wave length is easiest to arrange about the same magnitude with the smallest dimensions used in the system, or the wavelength is considerably longer, so that the close field model can be used. Later case the model used to the calculations may be even static field theory, just using preferably AC-magnetic fields for easier measurement.

The direction of magnetic field in middle of Helmholz coils is proximately parallel and uniform. This field is relatively easy to use for direction measurements with required accuracy. Using several pairs of Helmholz coils, it is possible to determine the angle of a coil with a good accuracy. Single coils may be used with worse accuracy. A rotating field can be used, for example 3 cols in 90-degree angles can produce a rotating field in 3 different perpendicular axles. The difficulty in this approach may be the difficulty to place the coils around the wanted space, where the measurement should take place. The signals may be synchronized with a separate signal, for example by a short magnetic or radio pulse in the beginning of a sinusoidal full wave of lower frequency. The synchronizing is not needed for each full wave. This enables a wireless receiver with coil to know the polarity of each field. Often the polarity information is not necessary, the direction is known before the measurement anyway.

The receiver and transmitter are interchangeable for the purpose of the invention throughout the document. This means, that even in the aforementioned radio positioning technologies all use stationary transmitters and receiver is calculating its position by measuring the phase or time difference between the signals from several transmitters, in case of implementing the invention there may be only one or two transmitters and several stationary receivers.

For example VORTAC is using transponder to measure the distance; the system according to the invention may use a cable instead of resending the radio signal. The synchronizing pulse like used in VORTAC may not be necessary at all, instead of that the timing can be determined by using a cable with a known delay to measure the phase of the turning or rotating radio transmission. And the “VORTAC beacon” may be the non-stationary sensor in order to measure the angle of the sensor from a single antenna. If a VORTAC-like measurement is used, the radio “beacon” is not necessary to send omni directional turning signal, only the necessary angle must be scanned.

For example an intra-oral wireless sensor needs to transmit at least the result of the measurement to the user of the X-ray system. That case it is may be easier to transmit the signal from inside the mouth to outside, and to measure from several receivers the timing or phase difference and to send the information to the outside by a transmitter. In this case the angle of the receiver can be detected by transmitting either directional signal and by measuring the minimum or maximum of the magnitude outside. The other way is to transmit several signals and to measure the phase difference in distant antenna and to calculate the angle from the phase differences i.e. from the distance differences of the transmitting antennas. The “VORTAC”-method may comprise a sensor with several microwave antennas that are sending phased FM-transmission to form a changing directional wave.

The sensor may for example transmit two harmonic signals from antennas of the different corners of the sensor. That case the single receiver in the direction of the X-ray source can calculate the angle of the X-ray receiver from the phase difference of the signals. The other way is to transmit the radio wave outside. If several radio waves of the same carrier frequency is used, the signals can be either sent in turns, or the signals may be sent in alternating phase, practically ending to two or more FM or Phase Modulated signals to a antenna array, the result being directional moving signal like the one used in VORTAC-navigation.

The invention is described also with reference to the following figures:

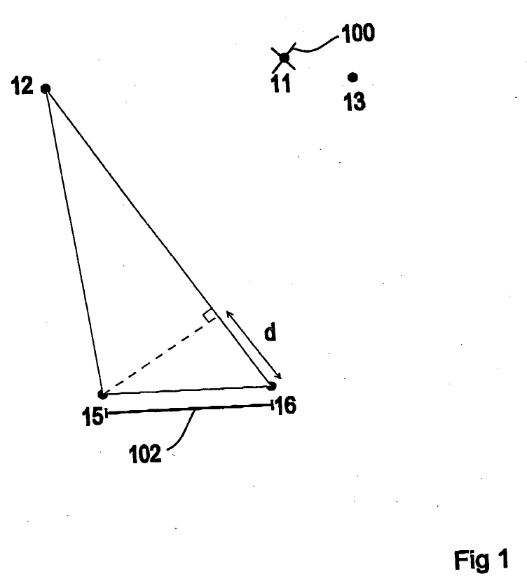

FIG. 1 shows a schematic figure of the principle of determination the position by measuring the traverse time and/or phase difference.

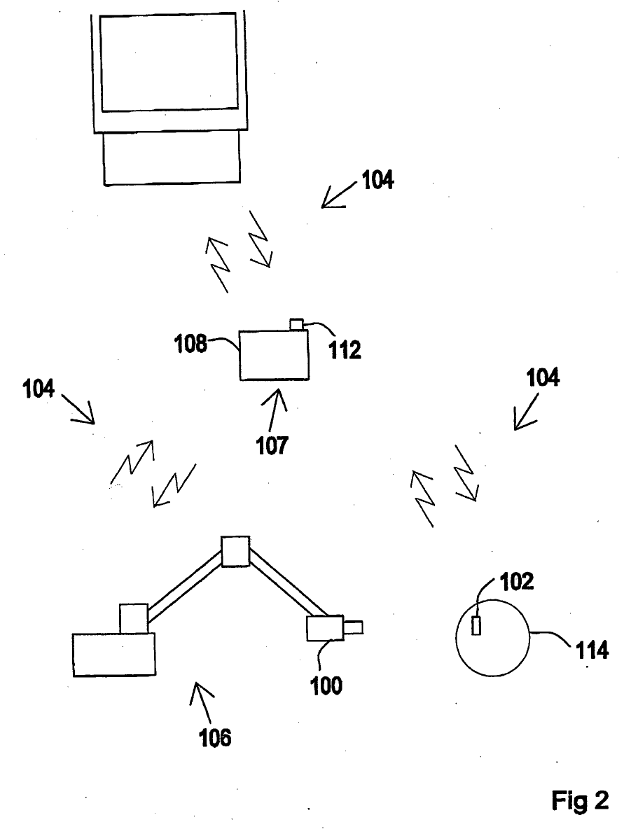

FIG. 2 shows an intraoral X-ray device arrangement presenting one preferred embodiment of the invention.

In FIG. 1 there are antennas 11, 12, 13 around the X-ray source 100. There are 2 antennas 15, 16 attached to the detector 102. Not necessarily all the antennas are drawn, to measure all the degrees of freedom minimum is 6 antennas and time-reference with cable, or an extra antenna for reference.

The radio signal is either transmitted or received by the antennas 11, 12, 13 the antennas 15, 16 receive or transmit the same signal. The differences d of distances 11 to 15, and 11 to 16 are calculated by measuring the phase difference of the signals. From the distance difference can be calculated the angle of the sensor with basic trigonometry. The radio signal may be about the same wave-length or longer or shorter than the distance between antennas 15 and 16. Much shorter wavelength causes difficulties with calculating multiple wavelengths in distance d, and the calculation result may be ambivalent. Much longer wavelength ends in very small differences in phase angles. With perpendicular signal the phase difference in the antennas 15 and 16 is zero with all the frequencies. This can be used to find perpendicular position for the X-ray source. The position is determined by the traverse time differences between the signals of antenna 11, 12, and 13. Three antennas are enough to determine the position, if there is a cable to send a time reference signal from to detector or to the detector. If the detector is wireless, there must be minimum 4 antennas in order to measure 3-dimensional position of the detector. The fourth antenna is needed for time and phase reference. Also instead of the cable the detector may comprise transponder, that responses to the signals from antennas 11, 12 and/or 13. The two-way traverse time is measured. The method may be the same as in Loran, with a difference that the frequency must be high to get enough resolution to the phase measurement. Another difference is that only one or two frequencies are needed, the transmitters may transmit in turns. And the phase difference of two antennas may be measured using waveguides between the measurement device and the antennas. The signal may be also modulated or even wide-spectrum signal; in that case the correlation is measured instead of the phase difference.

VORTAC principle is also easy to measure the position between two objects. The known VHF Omnidirectional Range navigation (VOR) is using a 360 degrees rotating signal. The method is widely used for aviation. The VORTAC is a beacon, with capability to measure the direction from the phase of rotating radio transmission beam; the phase is compared in the aeroplane to a separate synchronizing signal sent from the beacon. Synchronizing signal is sent when the rotating beam is for example towards the North. The distance is measured by transponder and measuring the two-way traverse time. The same principle can be used for measuring the position of an object by radio means. The wavelength should be smaller, so that smaller antennas can be used and the resolution is higher. Also there is no need for rotate the signal more than for a smaller angle, like 90 degrees. Also there should be two sets of antennas in order to get 3-dimensional information about the position. For example one antenna set turns the beam horizontally, and the other vertically. The receiver returns the signal with a cable or with a transmitter or a transponder is used.

The phase alternating antenna array for VORTAC-principle can be combined with the previously described measurement using the phase differences to measure the angle and position. Actually the VOR-principle can be derived from the previously described method, if the transmitting pair of antennas is changing the phase in order to make the transmitted beam change direction, and the receiver is measuring the amplitude phase of the turning beam. The transmission of turning beam by using phase alternating array of antennas can be implemented next to the X-ray sensor and the set of receivers outside are analysing the direction of the X-ray sensor by detecting the phase of the turning beam. By measuring the phase difference of the carrier from the transmitter, the position can be detected trigonometrically. If there is a cable available, the cable can be used for time reference.

The Helmholz coils or just single coils can be used to measure the wanted direction with any radio method for measuring the position. The benefit of magnetic measurement is relatively easy electronic design, but the coils may be unpractical compared to a set of microwave antennas.

In FIG. 2 is presented an intraoral X-ray device arrangement. It is important to notice that this is only an example of the medical X-ray device where the invention is possible to be utilized. The medical x-ray device in the embodiments of the invention is for example a dental panoramic X-ray device, a surgical C-arm X-ray, a mobile x-ray device or a mammography device.

In an intraoral x-ray device arrangement the articulated arm arrangement 106 moves the X-ray source 100 to the right position. The X-radiation begins by pressing the exposure button 112. The X-ray source 100 X-radiates the object 114, which is for example teeth of a patient. The detector 102 detects the X-radiation. The image information which is got by detecting the X-radiation is sent by communication link 104 to the computer 110. The computer comprises the software means to process the image information.

The X-ray device advantageously further comprises means for indicating to the user or the device, when the detector and X-ray source are in acceptable position or angle. The X-ray may also comprise a means for preventing to turn the X-ray source on when the detector in unacceptable position or angle. The indicating means may comprise a LED display or sound means to guide the user.

1. A method for determining the position of X-ray detector, characterized in that the detector is equipped with one or more radio or magnetic means, and one or more radio or magnetic means are in a distance from the detector, and said radio means are used to determine at least either position or angle of the detector in respect to the X-ray source.

2. A method according to claim 1, where the detector uses transmitter to transmit the signal and in the distance there are receivers that receive the signal, the phase or traverse time difference between the radio signal received in the receivers is used to determine the position or orientation

3. A method according to claim 1, where the detector uses receiver to detect the signals from distance, and the traverse time differences are used to calculate the position or orientation of the detector

4. A method according to the claim 1, where the detector is equipped with at least two antennas and the angle of the detector is determined from the phase difference or traverse time difference of the signals of the antennas.

5. A method according to the claim 1, where the desired direction is determined by using directional antenna or set of antennas.

6. A method according to the claim 1, where the desired direction is determined by using magnetic coils.

7. A X-ray device, characterized in that the X-ray detector is equipped with one or more radio or magnetic means, and one or more radio or magnetic means are in a distance from the detector, and said radio means are used to determine at least either position or angle of the detector in respect to the X-ray source.

8. A X-ray device according the claim 7 further comprising a means for indicating to the user or the device, when the detector and X-ray source are in acceptable position or angle.

9. A X-ray device according the claim 7 further comprising a means for preventing to turn the X-ray source on when the detector in unacceptable position or angle.
